(19) United States
(12) Patent Application Publication
(10) Pub. No.: US 2016/0122263 A1
(43) Pub. Date: May 5, 2016

(54) METHODS FOR ALKANE DEHYDROGENATION

(71) Applicant: SAUDI BASIC INDUSTRIES CORPORATION, Riyadh (SA)

(72) Inventors: Zeeshan H. Nawaz, Lahore (PK); Khaja Aliuddin Sharief, AP (IN); Faisal Baksh, Manama (BH); Abdullah Al-Qahtani, Riyadh (SA)

(21) Appl. No.: 14/891,963

(22) PCT Filed: May 29, 2014
(86) PCT No.: PCT/IB2014/061797

§ 371 Date: Nov. 17, 2015

Related U.S. Application Data
(60) Provisional application No. 61/829,780, filed on May 31, 2013.

(51) Int. Cl. C07C 5/333; B01J 23/42; B01J 23/26; B01J 21/04; B01J 23/14

(52) U.S. Cl. CPC C07C 5/3332; C07C 5/3337; B01J 21/04; B01J 23/14; B01J 23/26; B01J 23/42; C07C 2523/26; C07C 2523/42; C07C 2523/14; C07C 2521/04

(57) ABSTRACT

Disclosed herein are methods for dehydrogenation of alkanes to olefins by co-injecting the alkane feed with hydrogen. The present methods provide the improved feed conversion, desired product selectivity, total olefins in product stream, and lower catalyst deactivation rate.

### BACKGROUND OF THE INVENTION

Light olefins are an indispensable raw material for a wide range of petrochemical processes. The present non-oxidative processes, including direct dehydrogenation, suffer from needing a continuous heat supply (due to the endothermic reaction) and frequent catalyst regeneration. Direct alkane dehydrogenation (endothermic) is a process regularly employed for production of a variety of light alkenes or corresponding alkenes products, such as propane to propylene, iso-butane to iso-butylene, isooctane and alkylates. Moreover, direct dehydrogenation is useful for the production of high demand products, such as propylene or iso-butene. However, the effective reactor performance and process reliability largely depends upon the heat requirement for endothermic reaction. Although different catalytic dehydrogenation processes utilizing various catalysts have been commercialized, productivity problems still exist. Accordingly, there remains a need for improved bulk production methods for direct alkane dehydrogenation to olefin which can produce higher dehydrogenation yield and less frequent catalyst regeneration. This need and other needs are satisfied by the various aspects of the present disclosure.

### SUMMARY OF THE INVENTION

In accordance with the purposes of the invention, as embodied and broadly described herein, the invention relates, in one aspect, to a method for dehydrogenation of alkanes comprising: a) co-injecting a hydrocarbon feed stream comprising at least one C2 to C5 alkane with a hydrogen feed stream into a non-membrane dehydrogenation catalytic reactor comprising a chromium-based or platinum-based catalyst; and b) producing at least one dehydrogenated alkane product; wherein the reactor is run under non-oxidative conditions.

### BRIEF DESCRIPTION OF THE FIGURES

The accompanying figures, which are incorporated in and constitute a part of this specification, illustrate several aspects and together with the description serve to explain the principles of the invention.

FIG. 1 is a schematic drawing of a fluidized bed dehydrogenation system of the present invention.

FIG. 2 is a graph showing catalyst dehydrogenation performance with and without co-injection of hydrogen in accordance with the present invention.

Additional aspects of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited.

### A. DEFINITIONS

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification and in the claims which follow, reference will be made to a number of terms which shall be defined herein.

As used in the specification and the appended claims, the singular forms “a,” “an” and “the” include plural referents unless the context clearly dictates otherwise. Thus, for example, reference to “an olefin” includes mixtures of two or more paraffins or olefins.

As used herein, the term “combination” is inclusive of blends, mixtures, alloys, reaction products, and the like.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally substituted alkyl” means that the alkyl group can or cannot be substituted and that the description includes both substituted and unsubstituted alkyl groups.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspect or combination of aspects of the methods of the invention.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (“wt %”) of a component, unless specifically stated to the contrary, is based on the total weight of the formulation or composition in which the component is included. For example if a particular element or component in a composition or article is said to have 8% by weight, it is understood that this percentage is relative to a total compositional percentage of 100% by weight.

As used herein, the term or phrase “effective,” “effective amount,” or “conditions effective to” refers to such amount or condition that is capable of performing the function or property for which an effective amount is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

Compounds are described using standard nomenclature. For example, any position not substituted by any indicated group is understood to have its valence filled by a bond as indicated, or a hydrogen atom. A dash (“-”) that is not between two letters or symbols is used to indicate a point of attachment for a substituent. For example, —CHO is attached through carbon of the carbonyl group. Unless defined otherwise, technical and scientific terms used herein have the same meaning as is commonly understood by one of skill in the art to which this invention belongs.

The term “alkyl group” as used herein is a branched or unbranched saturated hydrocarbon group of 1 to 24 carbon atoms, such as methyl, ethyl, n propyl, isopropyl, n butyl, isobutyl, t butyl, pentyl, hexyl, heptyl, octyl, decyl, tetradecyl, hexadecyl, eicosyl, tetracosyl and the like. A “lower alkyl” group is an alkyl group containing from one to six carbon atoms.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein can be different from the actual publication dates, which can require independent confirmation.

### B. METHOD FOR DEHYDROGENATION OF ALKANES

As briefly described above, the present disclosure relates, in one aspect, to methods for dehydrogenation of alkanes. In other aspects, disclosure relates to bulk production methods for dehydrogenation of alkanes. In further aspects, disclosed is a method for dehydrogenation of alkanes comprising: a) co-injecting a hydrocarbon feed stream comprising at least one C2 to C5 alkane with a hydrogen feed stream into a non-membrane dehydrogenation catalytic reactor comprising a chromium-based or platinum-based catalyst; and b) producing at least one dehydrogenated alkane product; wherein the reactor is run under non-oxidative conditions.

In various aspects, the method comprises a hydrocarbon feed stream comprising at least one alkane. In further aspects, the alkane comprises at least one C2 to C5 alkane. In still further aspects, the alkane comprises ethane, propane, n-butane, isobutane, n-pentane, isopentane, or neopentane, or a combination thereof.

In further aspects, the dehydrogenated alkane product comprises at least one alkene or olefin. In still further aspects, the dehydrogenated alkane product comprises a corresponding dehydrogenated alkane product of a hydrocarbon present in the feed stream. In yet further aspects, the dehydrogenated alkane product is substantially free of water or carbon dioxide.

In further aspects, the dehydrogenated alkane product comprises ethene, propene, n-butene, iso-butene, n-pentene, or iso-pentene, or a combination thereof. In still further aspects, the dehydrogenated alkane product comprises but-1-ene, cis-2-butene, trans-2-butene, pent-1-ene, pent-2-ene, 2-methylbut-1-ene, 3-methylbut-1-ene, or 2-methyl-but-2-ene, or a combination thereof.

In various aspects, methods comprise co-injecting the hydrocarbon feed stream with hydrogen. Without wishing to be bound by a particular theory, it is generally believed that hydrogen addition may adversely affect olefin yield by directing the reaction stoichiometrically away from dehydrogenation and toward olefin saturation. However, it was surprisingly found that by co-injecting hydrogen, and in certain more specific aspects, by increasing the hydrogen to hydrocarbon feed molar ratio to a controlled molar ratio, the desired product selectivity and feed conversion can be increased.

In further aspects, the hydrogen feed stream and hydrocarbon feed stream has a hydrogen to hydrocarbon molar ratio in the range of from about 0.01 to about 0.5, including exemplary values of 0.02, 0.03, 0.04, 0.05, 0.06, 0.07, 0.08, 0.09, 0.1, 0.15, 0.2, 0.25, 0.3, 0.35, 0.4, and 0.45. In still further aspects, the pressure can be in a range derived from any of the two above listed exemplary values. For example, the molar ratio can range from 0.1 to 0.5 or from 0.1 to 0.4. In yet further aspects, the hydrogen feed stream and hydrocarbon feed stream is substantially free of oxygen, air, or steam.

In various aspects, the hydrogen feed stream is contacted with the hydrocarbon feed stream prior to, during, or after preheating of the hydrocarbon feed stream, or a combination thereof. In one aspect, the hydrogen feed stream is contacted with the hydrocarbon stream prior to preheating of the hydrocarbon feed stream. In other aspects, the hydrogen feed stream is contacted with the hydrocarbon stream during preheating of the hydrocarbon feed stream. In further aspects, the hydrogen feed stream is contacted with the hydrocarbon stream after preheating of the hydrocarbon feed stream.

In some aspects, the hydrogen feed stream comprises virgin hydrogen from a hydrogen plant, or recycled hydrogen from a hydrogen-recovery process, or a combination thereof. For example, the recycled hydrogen is recovered from a hydrogen-recovery unit. In further aspects, the hydrogen-recovery unit is attached to the dehydrogenation product stream.

In various aspects, the hydrogen stream is contacted with the hydrocarbon stream prior to a catalytic reactor, in one or more catalytic reactors, or between catalytic reactors, or a combination thereof. In one aspect, the hydrogen feed stream is contacted with the hydrocarbon feed stream prior to a catalytic reactor. In other aspects, the hydrogen feed stream is contacted with the hydrocarbon feed stream in one or more catalytic reactors. In further aspects, the hydrogen feed stream is contacted with the hydrocarbon feed stream between catalytic reactors. In still further aspects, the hydrogen feed stream is introduced into the system at point prior to a catalytic reactor, directly into a catalytic reactor, or between catalytic reactors, or a combination thereof.

In further aspects, the method comprises single catalytic reactors, or multiple catalytic reactors in one train, or a combination thereof. In some aspects, the catalytic reactor comprises at least one catalytic dehydrogenation reaction zone. In other aspects, the catalytic reactor comprises multiple catalytic dehydrogenation reaction zones. In still further aspects, the method comprises multiple catalytic reactors.

In other aspects, the hydrogen feed stream is contacted with a hydrocarbon feed stream prior to introduction to a catalytic dehydrogenation reaction zone, in one or more catalytic dehydrogenation reaction zones, or between catalytic dehydrogenation reaction zones, or combinations thereof. In one aspect, the hydrogen feed stream is contacted with a hydrocarbon feed stream prior to introduction to a catalytic dehydrogenation reaction zone. In another aspect, the hydrogen feed stream is contacted with a hydrocarbon feed stream in one or more catalytic dehydrogenation reaction zones. In yet another aspect, the hydrogen feed stream is contacted with a hydrocarbon feed stream between catalytic dehydrogenation reaction zones.

In further aspects, the hydrogen feed stream and hydrocarbon feed stream is directly contacted with a catalyst or catalyst bed. In still further aspects, the hydrogen feed stream and hydrocarbon feed stream is contacted with a catalyst or a catalyst bed in an upward, downward, or radial flow direction, or a combination thereof. In one aspect, the hydrogen feed stream and hydrocarbon feed stream is contacted with a catalyst or a catalyst bed in an upward flow direction. In another aspect, the hydrogen feed stream and hydrocarbon feed stream is contacted with a catalyst or a catalyst bed in a downward flow direction. In yet another aspect, the hydrogen feed stream and hydrocarbon feed stream is contacted with a catalyst or a catalyst bed in a radial flow direction.

In further aspects, the hydrogen feed stream and hydrocarbon feed stream is in a liquid phase, or a vapor phase, or a combination thereof. In one aspect, the hydrogen feed stream and hydrocarbon feed stream is in a liquid phase. In another aspect, the hydrogen feed stream and hydrocarbon feed stream is in a vapor phase.

In various aspects, the catalytic reactor comprises a fixed bed reactor, a multi-tubular reactor, a fluidized bed reactor, a moving bed reactor, or a combination thereof. In one aspect, the catalytic reactor comprises a fixed bed reactor. In another aspect, the catalytic reactor comprises a multi-tubular reactor. In yet another aspect, the catalytic reactor comprises a fluidized bed reactor. In still another aspect, the catalytic reactor comprises a moving bed reactor.

In further aspects, the catalytic reactor comprises at least one catalyst regeneration unit. In other aspects, the catalytic reactor comprises multiple catalyst regeneration units. In further aspects, the catalyst is regenerated by burning coke on the catalyst surface at a temperature greater than the average temperature of the reactor in a stream of steam, air, oxygen, and fuel gas. The catalyst regeneration is an exothermic process, and can act as a source of heat for the catalyst and to maintain the reactor temperature. The regeneration residence time can depend on the type of catalyst, catalyst loading, and the circulation rate required for the steady operation in the reactor. The fuel gas injection requirement can vary with the severity and the mode of operation. The catalyst regeneration parameters further influence the temperature requirement for catalyst regeneration. As such, the temperature for catalyst regeneration can range from 550° C. to 750° C., including exemplary values of 575° C., 600° C., 625° C., 650° C., 675° C., 700° C., and 725° C. In still further aspects, the temperature for catalyst regeneration can be in a range derived from any two of the above listed exemplary temperatures. For example, the temperature for catalyst regeneration can range from 575° C. to 725° C.

In further aspects, at least a portion of the hydrogen formed or released during a reaction step is recovered. In still further aspects, a portion of the recovered hydrogen is reintroduced back into the system. In some aspects, at least a portion of the recovered hydrogen is recovered for an external facility. In other aspects, at least a portion of the recovered hydrogen is reintroduced into the hydrogen stream for dehydrogenation. In further aspects, at least a portion of the recovered hydrogen is used as a stripping gas. In still further aspects, when the catalytic reactor is a fluidized or moving bed reactor, at least a portion of the hydrogen formed or released during a reaction step is recovered and used as a catalyst lift gas.

In further aspects, the catalytic reactor comprises various reactor conditions and operating parameters. For example, in some aspects, the reactor is run under substantially non-oxidative conditions. In other aspects, wherein the reactor is run under fully non-oxidative conditions. In some aspects, the alkane dehydrogenation reaction is measured using reactor conditions or operating parameters. For example, according to some aspects, the reactor conditions comprise a feed stream having a weight hourly space velocity (WHSV) of from about 1 hr⁻¹ to about 10000 hr⁻¹. As used herein, WHSV refers to the weight hourly space velocity and allows for relating the feed stream flow rate to the reactor volume. WHSV indicates how many reactor volumes of feed can be treated in a unit time, and it is commonly regarded as the reciprocal of the reactor space time. These operation parameters vary from feed to feed (or feed composition), catalyst to catalyst (or active metal loading content), mode of operation, and size of design (desired hydrodynamics).

In some aspects, the alkane is dehydrogenated in the presence of a Cr-based catalyst at a WHSV ranging from 100 inverse hours (h⁻¹) to 1000 h⁻¹, including exemplary values of 200 h⁻¹, 300 h⁻¹, 400 h⁻¹, 500 h⁻¹, 600 h⁻¹, 700 h⁻¹, 800 h⁻¹, and 900 h⁻¹. In still further aspects, the WHSV can be in a range derived from any two of the above listed exemplary WHSV values. For example, the WHSV can be 100 h⁻¹ to 500 h⁻¹ or from 200 h⁻¹ to 400 h⁻¹. In one aspect, the alkane is dehydrogenated in the presence of Cr₂O₃/Al₂O₃. In a further aspect, the Cr loading can range from range of 5 wt % to 40 wt %.

In other aspects, the alkane is dehydrogenated in the presence of a Pt-based catalyst at a WHSV ranging from 1 h⁻¹ to 50 h⁻¹, including exemplary values of 2 h⁻¹, 3 h⁻¹, 4 h⁻¹, 5 h⁻¹, 6 h⁻¹, 7 h⁻¹, 8 h⁻¹, 9 h⁻¹, 10 h⁻¹, 11 h⁻¹, 12 h⁻¹, 13 h⁻¹, 14 h⁻¹, 15 h⁻¹, 16 h⁻¹, 17 h⁻¹, 18 h⁻¹, 19 h⁻¹, 20 h⁻¹, 25 h⁻¹, 30 h⁻¹, 35 h⁻¹, 40 h⁻¹, and 45 h⁻¹. In still further aspects, the WHSV can be in a range derived from any two of the above listed exemplary WHSV values. For example, the WHSV range from 1 h⁻¹ to 30 h⁻¹, or from 1 h⁻¹ to 20 h⁻¹ for a Pt-based catalyst.

As used herein, residence time refers to the average amount of time that the reacting catalyst spends in the reactor system. The residence time of the catalyst can also be called the catalyst circulation rate.

In further aspects, the catalyst circulation rate (residence time of the catalyst) in the fluidized bed regenerator column, for the Cr-based catalyst ranges from 2 minutes to 22 minutes, including exemplary values of 5 minutes, 7 minutes, 10 minutes, 13 minutes, 15 minutes, 17 minutes, and 20 minutes. In still further aspects, the catalyst circulation rate can be in a range derived from any two of the above listed exemplary catalyst circulation rate values. For example, the catalyst circulation rate can range from 5 minutes to 20 minutes.

In one aspect, the catalyst circulation rate (residence time of the catalyst) in the fluidized bed regenerator column, for the Pt-based catalyst ranges from 1 hour to 8 hours, including exemplary values of 1.5 hour (hr), 2 hr, 2.5 hr, 3 hr, 3.5 hr, 4 hr, 4.5 hr, 5 hr, 5.5 hr, 6 hr, 6.5 hr, 7 hr, and 7.5 hr. In still further aspects, the catalyst circulation rate can be in a range derived from any two of the above listed exemplary catalyst circulation rate values. For example, the catalyst circulation rate can range from 1.5 hours to 6.5 hours or from 2.5 hours to 7.5 hours.

In further aspects, the reactor conditions comprise a pressure ranging from 0.1 atmophere (atm) to 3 atm, including exemplary values of 0.2 atm, 0.3 atm, 0.4 atm, 0.5 atm, 0.6 atm, 0.7 atm, 0.8 atm, 0.9 atm, 1 atm, 1.2 atm, 1.4 atm, 1.6 atm, 1.8 atm, 2 atm, 2.2 atm, 2.4 atm, 2.6 atm, and 2.8 atm. In still further aspects, the pressure can be in a range derived from any of the two above listed exemplary pressures. For example, the pressure can range from 0.2 atm to 2.8 atm or from 0.5 atm to 2.5 atm.

In further aspects, the reactor conditions comprise a temperature ranging from 450° C. to 700° C., including exemplary values of 455° C., 460° C., 465° C., 470° C., 475° C., 480° C., 485° C., 490° C., 495° C., 500° C., 505° C., 510° C., 515° C., 520° C., 525° C., 530° C., 535° C., 540° C., 545° C., 550° C., 555° C., 560° C., 565° C., 570° C., 575° C., 580° C., 585° C., 590° C., 595° C., 600° C., 605° C., 610° C., 615° C., 620° C., 625° C., 630° C., 635° C., 640° C., 645° C., 650° C., 655° C., 660° C., 665° C., 670° C., 675° C., 680° C., 685° C., 690° C., 695° C. and 700° C. In still further aspects, the temperature can be in a range derived from any two of the above listed exemplary temperatures. For example, the temperature can range from 500° C. to 640° C. or from 500° C. to 600° C.

In various aspects, the methods of the present invention comprise at least one catalyst. In further aspects, the catalyst comprises a chromium-based catalyst, a platinum-based catalyst, or a combination thereof. In some aspects, the catalyst is a chromium-based catalyst. In other aspects, the catalyst is a platinum-based catalyst. In one aspect, the alkane is dehydrogenated in the presence of Cr₂O₃/Al₂O₃. In another aspect, the alkane is dehydrogenated in the presence of Pt-Sn/SAPO-34. In other aspects, modified catalyst are used. For example, according to further aspects of the inventions, the modified catalysts can include, but are not limited to platinum aluminum supported, platinum on zinc aluminate, platinum on zeolite support, for example, ZSM-5 or SBA-15, and cromium oxide based catalysts further modified with promoters and aluminum or zeolite supports.

In further aspects, the catalyst is used with a carrier or support material. In still further aspects, the support material comprises a molecular sieve support or mesh. In yet further aspects, the support material comprises modified zeolites or alumina oxide, or modified alumina oxide. In some aspects, the support material comprises inert materials. In other aspects, the support or carrier material is physically mixed with or interspersed among the heat-generating material. In further aspects, the support or carrier material is capable of supporting heat-generating inert materials.

In further aspects, the catalyst is used with a carrier or support material. In still further aspects, the support material comprises a molecular sieve support or mesh. In some aspects, the support material comprises inert materials, for example, SiO₂. In some aspects, the support or carrier material is capable of supporting heat-generating inert materials. In other aspects, the support or carrier material is physically mixed with heat-generating material. In further aspects, the catalyst is loaded on the carrier or support.

In further aspects, the carrier or support material can withstand a temperature of at least about 450° C., including exemplary values of 455° C., 460° C., 465° C., 470° C., 475° C., 480° C., 485° C., 490° C., 495° C., 500° C., 505° C., 510° C., 515° C., 520° C., 525° C., 530° C., 535° C., 540° C., 545° C., 550° C., 555° C., 560° C., 565° C., 570° C., 575° C., 580° C., 585° C., 590° C., 595° C., 600° C., 605° C., 610° C., 615° C., 620° C., 625° C., 630° C., 635° C., 640° C., 645° C., 650° C., 655° C., 660° C., 665° C., 670° C., 675° C., 680° C., 685° C., 690° C., 695° C. and 700° C. In still further aspects, the temperature can be in a range derived from any two of the above listed exemplary temperatures. For example, the temperature can range from 500° C. to 640° C. or from 500° C. to 600° C.

In further aspects, the catalyst comprises more than one layer or component. For example, the catalyst can further comprise a secondary component comprising any heat-generating inert material physically mixed or layered with the catalyst and a carrier capable of supporting the heat-generating inert material.

In further aspects, the heat generating material comprises at least 10 wt % of the catalyst bed. In yet further aspects, the heat generating material comprises at least 25 wt % of the catalyst bed. In still further aspects, the heat generating material comprises at least 50 wt % of the catalyst bed. In even further aspects, the heat generating material comprises at least 60 wt % of the catalyst bed.

In some aspects, the support material comprises alumina, silica-alumina, aluminum oxide, monohydrates, silica or silicates, hydrotalcites, zeolites, or a combination thereof. In other aspects, the support material comprises alumina, silica-alumina, or zeolite. In further aspects, the support material comprises modified zeolites, alumina oxide, or modified alumina oxide.

In further aspects, the catalyst is modified by a promoter. In still further aspects, the catalyst comprises an active component selected from a metal oxide of Group 4, Group 5, or Group 6, or a combination thereof. Exemplary metals include, but are not limited to Ca, K, Fe, Cu, Co, Mn, Ru, Rh, Pd, Si, Ti, Na, Zr, Al, Mg, and W.

In further aspects, the Cr loading can range from 5 wt % to 40 wt %, including exemplary values of 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 35 wt %, 36 wt %, 37 wt %, 38 wt %, or 39 wt %. In still further aspects, the Cr loading can be in a range derived from any two of the above listed exemplary Cr loading values. For example, the Cr loading can range from 9 wt % to 21 wt %. In yet further aspects, the Cr loading can be with multiple promoters in a range derived from any two of the above listed exemplary Cr loading value. In some aspects, the catalyst comprises chromium oxide based catalyst modified with promoters. In other aspects, the catalyst comprises chromium oxide based catalyst modified with at least one promoter supported on aluminum support or zeolite support.

In further aspects, the Pt loading can range from 0.1 wt % to 4 wt %, including exemplary values of 0.2 wt %, 0.3 wt %, 0.4 wt %, 0.5 wt %, 0.6 wt %, 0.7 wt %, 0.8 wt %, 0.9 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt %, 2.5 wt %, 2.6 wt %, 2.7 wt %, 2.8 wt %, 2.9 wt %, 3.0 wt %, 3.1 wt %, 3.2 wt %, 3.3 wt %, 3.4 wt %, 3.5 wt %, 3.6 wt %, 3.7 wt %, 3.8 wt %, or 3.9 wt %. In still further aspects, the Pt loading can be in a range derived from any two of the above listed exemplary Pt loading values. For example, the Pt loading can range from 0.1 wt % to 3.0 wt %. In yet further aspects, the Pt loading can be with multiple promoters in a range derived from any two of the above listed exemplary Pt loading value. In some aspects, the catalyst comprises a platinum-based catalyst supported on aluminum support, a platinum-based catalyst supported on zinc aluminate support, or a platinum-based catalyst supported on zeolite support, for example, ZSM5 or SBA-15.

In other aspects, a platinum-based catalyst or a chromium-based catalyst can be supported on a non-acid support, such as alumina or silica alumina. In a further aspect, a platinum-based catalyst or a chromium based catalyst can be supported on an acid support, a zeolite, or a metal oxide, or a combination thereof. In another aspect, the methods can utilize a multi-component catalyst and/or a metal oxide. For example, the platinum or the chromium based catalysts on a non-acid support, an acid support, a zeolite, or a metal oxide, or a combination thereof, are recited in U.S. Pat. Nos. 5,132,484; 3,488,402; 2,374,404; 8,063,261 B2; 4,677,237 A; 4,716,143; 4,762,960 A; 289,137; 4973779 A; EP1,147,810 A1; and International Publication No. WO/2005/040075, all of which are hereby incorporated in their entirety for the specific purpose of disclosing various platinum or chromium based catalysts on a non-acid support compositions and methods. In a further aspect, the invention has the ability to use any catalyst appropriate for direct dehydrogenation.

In one aspect, the catalyst can be modified by one or more promoters. The promoter can control the stereochemistry of the dehydrogenation reaction. For example, catalysts modified by one or more promoters are recited in U.S. Pat. Nos. 5,198,597; 5,146,034; 3,899,544; 3,679,773; 4,000,210; 2,374,404; 8,101,541 B2: 4,677,237 A; 2,945,823; 7012038 B2; 4,762,960 A; 4,177,218; 2,814,599; 3,679,773; GB 942,944; CN 200910091226 and PK 140812/2010, all of which are hereby incorporated in their entirety for the specific purpose of disclosing various compositions and methods of catalysts modified by one or more promoters.

In another aspect, the catalyst comprises one or more promoters dispersed on aluminum oxide, silicon oxide, or zeolite, or combination thereof. For example, the catalysts comprising one or more promoters dispersed on aluminum oxide, silicon oxide, or zeolite, other metal oxides, or combination thereof are recited in U.S. Pat. Nos. 2,814,599; 3,679,773; 5,416,052; 5,146,034; 3,507,931; 3,551,353; 3,932,554; 4,935,578; and 5,132,479; and CN Pat. No. 1,762,931, all of which are hereby incorporated in their entirety for the specific purpose of disclosing various compositions and methods of catalysts comprising one or more promoters.

As briefly described above, the present methods can improve overall reactor performance and reduce fuel gas consumption by co-injecting hydrogen at various process stages. For example, when fluidized bed reactors are utilized, the catalyst is directly fluidized within the hydrocarbon feedstock. The product stream is subsequently separated from the catalyst, and any spent catalyst is directed back to the stripping zone and catalyst regenerator for regeneration. In various aspects, the catalyst regeneration involves coke removal from the catalyst, requiring stripping gas. In a further aspect, the fresh catalyst will also require catalyst lift gas to return regenerated catalyst to the reactor. Typically, fluidized bed reactors require a large amount of lift gas for the catalyst transfer from one zone to another zone. This lift gas can affect the overall performance of the catalyst adversely and can increase operational costs.

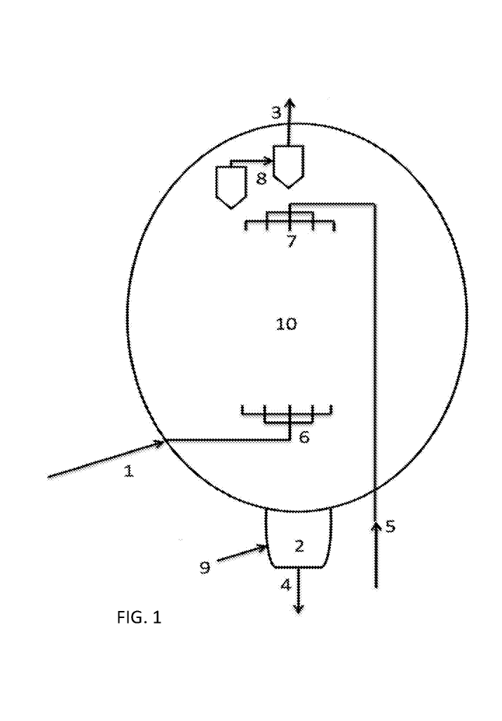

In one aspect, FIG. 1 shows an exemplary fluidized bed dehydrogenation system employing a method of the present invention. A co-injected feed stream comprising one or more alkanes and hydrogen enters through line 1 to reaction zone 10, through the feed distributor 6. In the present embodiment, the reactor is divided in two zones: reaction zone 10 and the stripping zone section 2. In some aspects, both the reaction zone and stripping zone are equipped with an internal grid to achieve superior gas solid contact. The dehydrogenation product gas 3 is separated from the catalyst in a series of primary and secondary cyclones 8. Additionally, from the stripping zone 2, the catalyst is shifted to the catalyst regenerator 4. In some aspects, in the stripping zone 2, the stripping gas 9 is co-injected with hydrogen. Fresh regenerated catalyst is pushed by the catalyst lift gas 5, and rushed to the catalyst distributor 7. In some aspects, the catalyst lift gas 5 comprises recovered hydrogen produced during a reaction step. In other aspects, the catalyst lift gas 5 can be supplemented with another gas in the event the lift gas demand exceeds the recovered hydrogen, for example, with supplemental natural gas.

In various aspects, the disclosed methods exhibit various advantages over alkane dehydrogenation methods of the prior art. In at least one aspect, the present method produces an improved hydrocarbon feed conversion. In a further aspect, the present method produces an improved selectivity to the corresponding alkene from an alkane present in the hydrocarbon feed stream.

In at least one aspect, the present method exhibits a lower catalyst deactivation rate when compared to a reference method consisting of substantially identical components except for the absence of the hydrogen stream and reacted under substantially identical conditions.

In further aspects, the present method exhibits a reduced frequency of catalyst regeneration when compared to a reference method consisting of substantially identical components except for the absence of the hydrogen stream and reacted under substantially identical conditions.

In further aspects, the present method exhibits an increased olefin yield when compared to a reference method consisting of substantially identical components except for the absence of the hydrogen stream and reacted under substantially identical conditions.

In further aspects, the present method exhibits increased olefin selectivity when compared to a reference method consisting of substantially identical components except for the absence of the hydrogen stream and reacted under substantially identical conditions.

In further aspects, the catalysts employed in the present method exhibit higher active sites stability during the course of reaction and continuously reduce the active metal sites and maintains catalytic activity without contributing to secondary reactions.

### C. ASPECTS

In various aspects, the present invention pertains to and includes at least the following aspects.

Aspect 1: A method for dehydrogenation of an alkane comprising: (a) co-injecting a hydrogen stream with a hydrocarbon stream comprising at least one C2 to C5 alkane into a non-membrane dehydrogenation catalytic reactor comprising a chromium-based catalyst or platinum-based catalyst; and (b) producing at least one dehydrogenated alkane product; wherein the reactor is run under substantially non-oxidative conditions.

Aspect 2: The method of aspect 1, wherein the at least one dehydrogenated product comprises a corresponding alkene product.

Aspect 3: The method according to any preceding aspect, wherein the hydrocarbon feed comprises ethane, propane, n-butane, isobutane, pentane, isopentane, or neopentane, or a combination thereof.

Aspect 4: The method according to any preceding aspect, wherein the dehydrogenated alkane product comprises at least one alkene or olefin.

Aspect 5: The method according to any preceding aspect, wherein the dehydrogenated alkane product comprises a corresponding dehydrogenated alkane product of a hydrocarbon present in the feed stream.

Aspect 6: The method according to any preceding aspect, wherein the dehydrogenated alkane product comprises ethene, propene, n-butene, iso-butene, n-pentene, or iso-pentene, or a combination thereof.

Aspect 7: The method according to any preceding aspect, wherein the dehydrogenated alkane product comprises but-1-ene, cis-2-butene, trans-2-butene, pent-1-ene, pent-2-ene, 2-methylbut-1-ene, 3-methylbut-1-ene, or 2-methyl-but-2-ene, or a combination thereof.

Aspect 8: The method according to any preceding aspect, wherein the dehydrogenated alkane product is substantially free of water or carbon dioxide.

Aspect 9: The method according any preceding aspect, wherein the reactor is run under fully non-oxidative conditions.

Aspect 10: The method according to any preceding aspect, wherein the hydrogen feed stream and hydrocarbon feed stream has a hydrogen to hydrocarbon molar ratio in the range of from about 0.01 to about 0.5.

Aspect 11: The method according to any preceding aspect, wherein the hydrogen feed stream and hydrocarbon feed stream has a hydrogen to hydrocarbon molar ratio in the range of from about 0.1 to about 0.4.

Aspect 12: The method according to any preceding aspect, wherein the hydrogen feed stream and hydrocarbon feed stream is substantially free of oxygen, air, or steam.

Aspect 13: The method according to any preceding aspect, wherein the hydrogen feed stream is contacted with the hydrocarbon stream prior to preheating of the hydrocarbon feed stream.

Aspect 14: The method according to any preceding aspect, wherein the hydrogen feed stream is contacted with the hydrocarbon stream during preheating of the hydrocarbon feed stream.

Aspect 15: The method according to any preceding aspect, wherein the hydrogen feed stream is contacted with the hydrocarbon stream after preheating of the hydrocarbon feed stream.

Aspect 16: The method according to any preceding aspect, wherein the hydrogen feed stream comprises virgin hydrogen from a hydrogen plant, or recycled hydrogen from a hydrogen-recovery process, or a combination thereof.

Aspect 17: The method according to any preceding aspect, wherein the hydrogen-recovery unit is attached to the dehydrogenation product stream.

Aspect 18: The method according to any preceding aspect, the hydrogen stream is contacted with the hydrocarbon stream prior to a catalytic reactor, in one or more catalytic reactors, or between catalytic reactors, or a combination thereof.

Aspect 19: The method according to any preceding aspect, the hydrogen feed stream is contacted with the hydrocarbon feed stream prior to a catalytic reactor.

Aspect 20: The method according to any preceding aspect, the hydrogen feed stream is contacted with the hydrocarbon feed stream in one or more catalytic reactors.

Aspect 21: The method according to any preceding aspect, the hydrogen feed stream is contacted with the hydrocarbon feed stream between catalytic reactors.

Aspect 22: The method according to any preceding aspect, wherein the hydrogen feed stream is introduced into the system at point prior to a catalytic reactor, directly into a catalytic reactor, or between catalytic reactors, or a combination thereof.

Aspect 23: The method according to any preceding aspect, wherein the method comprises single catalytic reactors, or multiple catalytic reactors in one train, or combinations thereof.

Aspect 24: The method according to any preceding aspect, wherein the catalytic reactor comprises at least one catalytic dehydrogenation reaction zone.

Aspect 25: The method according to any preceding aspect, wherein the catalytic reactor comprises multiple catalytic dehydrogenation reaction zones.

Aspect 26: The method according to any preceding aspect, wherein the method comprises multiple catalytic reactors.

Aspect 27: The method according to any preceding aspect, wherein the catalytic reactor comprises a fixed bed reactor, a multi-tubular reactor, a fluidized bed reactor, a moving bed reactor, or a combination thereof.

Aspect 28: The method according to any preceding aspect, wherein the catalytic reactor comprises a fixed bed reactor.

Aspect 29: The method according to any preceding aspect, wherein the catalytic reactor comprises a multi-tubular reactor.

Aspect 30: The method according to any preceding aspect, wherein the catalytic reactor comprises a fluidized bed reactor.

Aspect 31: The method according to any preceding aspect, wherein the catalytic reactor comprises a moving bed reactor.

Aspect 32: The method according to any preceding aspect, wherein the catalytic reactor comprises at least one catalyst regeneration unit.

Aspect 33: The method according to any preceding aspect, wherein the catalytic reactor comprises multiple catalyst regeneration units.

Aspect 34: The method according to any preceding aspect, wherein the hydrogen feed stream is contacted with a hydrocarbon feed stream prior to introduction to a catalytic dehydrogenation reaction zone, in one or more catalytic dehydrogenation reaction zones, or between catalytic dehydrogenation reaction zones, or combinations thereof.

Aspect 35: The method according to any preceding aspect, wherein the hydrogen feed stream is contacted with a hydrocarbon feed stream prior to introduction to a catalytic dehydrogenation reaction zone.

Aspect 36: The method according to any preceding aspect, wherein the hydrogen feed stream is contacted with a hydrocarbon feed stream in one or more catalytic dehydrogenation reaction zones.

Aspect 37: The method according to any preceding aspect, wherein the hydrogen feed stream is contacted with a hydrocarbon feed stream between catalytic dehydrogenation reaction zones.

Aspect 38: The method according to any preceding aspect, wherein reactor conditions comprise maintaining a feed stream having a weight hourly space velocity (WHSV) of from about 1 hr⁻¹ to about 1000 hr⁻¹.

Aspect 39: The method according to any preceding aspect, wherein reactor conditions comprise maintaining a pressure ranging from 0.1 atm to 3 atm.

Aspect 40: The method according to any preceding aspect, wherein reactor conditions comprise maintaining a temperature ranging from 490° C. to 700° C.

Aspect 41: The method according to any preceding aspect, wherein the catalyst is a chromium-based catalyst, and reactor conditions comprise maintaining a feed stream having a liquid hourly space velocity (LHSV) of from about 200 hr⁻¹ to about 500 hr⁻¹.

Aspect 42: The method according to any preceding aspect, wherein the catalyst is a platinum-based catalyst, and reactor conditions comprise maintaining a feed stream having a weight hourly space velocity (WHSV) of from about 1 hr⁻¹ to about 20 hr⁻¹.

Aspect 43: The method according to any preceding aspect, wherein the catalyst comprises a chromium-based catalyst.

Aspect 44: The method according to any preceding aspect, wherein the catalyst comprises a platinum-based catalyst.

Aspect 45: The method of according to any preceding aspect, wherein the chromium-based catalyst comprises in the range of 5 wt % to about 40 wt % loading.

Aspect 46: The method according to any preceding aspect, wherein the platinum-based catalyst comprises in the range of from 0.1 to about 3 wt % loading.

Aspect 47: The method according to any preceding aspect, wherein the catalyst comprises an active component selected from a metal oxide of Group 4, Group 5, or Group 6 of the periodic table, or a combination thereof.

Aspect 48: The method according to any preceding aspect, wherein the catalyst further comprises a carrier or support material.

Aspect 49: The method according to any preceding aspect, wherein the support material comprises a molecular sieve support.

Aspect 50: The method according to any preceding aspect, wherein the support material comprises inert materials.

Aspect 51: The method according to any preceding aspect, wherein the support material comprises alumina, silica-alumina, aluminum oxide, monohydrates, silica or silicates, hydrotalcites, zeolites, or a combination thereof.

Aspect 52: The method according to any preceding aspect, wherein the support material comprises alumina, silica-alumina, or zeolite.

Aspect 53: The method according to any preceding aspect, wherein the catalyst is present in a catalyst bed.

Aspect 54: The method according to any preceding aspect, wherein the catalyst is physically interspersed with the inert materials, or located between inert materials.

Aspect 55: The method according to any preceding aspect, wherein the catalyst bed comprises heat generating material.

Aspect 56: The method according to any preceding aspect, wherein the heat generating material is interspersed among or inserted between inert materials.

Aspect 57: The method according to any preceding aspect, wherein the heat generating material forms a layer on top, bottom, or in the center of the catalyst bed.

Aspect 58: The method according to any preceding aspect, wherein the heat generating material comprises at least 10 wt % of the catalyst bed.

Aspect 59: The method according to any preceding aspect, wherein the heat generating material comprises at least 25 wt % of the catalyst bed.

Aspect 60: The method according to any preceding aspect, wherein the heat generating material comprises at least 50 wt % of the catalyst bed.

Aspect 61: The method according to any preceding aspect, wherein the heat generating material comprises at least 60 wt % of the catalyst bed.

Aspect 62: The method according to any preceding aspect, wherein the hydrogen feed stream and hydrocarbon feed stream is directly contacted with a catalyst or catalyst bed.

Aspect 63: The method according to any preceding aspect, wherein the hydrogen feed stream and hydrocarbon feed stream is contacted with a catalyst or a catalyst bed in an upward, downward, or radial flow direction, or a combination thereof.

Aspect 64: The method according to any preceding aspect, wherein the hydrogen feed stream and hydrocarbon feed stream is contacted with a catalyst or a catalyst bed in an upward flow direction.

Aspect 65: The method according to any preceding aspect, wherein the hydrogen feed stream and hydrocarbon feed stream is contacted with a catalyst or a catalyst bed in a downward flow direction.

Aspect 66: The method according to any preceding aspect, wherein the hydrogen feed stream and hydrocarbon feed stream is contacted with a catalyst or a catalyst bed in a radial flow direction.

Aspect 67: The method according to any preceding aspect, wherein the the hydrogen feed stream and hydrocarbon feed stream is in a liquid phase, or a vapor phase, or a combination thereof.

Aspect 68: The method according to any preceding aspect, wherein the hydrogen feed stream and hydrocarbon feed stream is in a vapor phase.

Aspect 69: The method according to any preceding aspect, wherein the method produces an improved hydrocarbon feed conversion.

Aspect 70: The method according to any preceding aspect, wherein the method produces an improved selectivity to a corresponding alkene of a hydrocarbon present in the feed stream.

Aspect 71: The method according to any preceding aspect, wherein the method exhibits a lower catalyst deactivation rate when compared to a reference method consisting of substantially identical components except for the absence of the hydrogen stream and reacted under substantially identical conditions.

Aspect 72: The method according to any preceding aspect, wherein the method exhibits a reduced frequency of catalyst regeneration when compared to a reference method consisting of substantially identical components except for the absence of the hydrogen stream and reacted under substantially identical conditions.

Aspect 73: The method according to any preceding aspect, wherein the method exhibits an increased olefin yield when compared to a reference method consisting of substantially identical components except for the absence of the hydrogen stream and reacted under substantially identical conditions.

Aspect 74: The method according to any preceding aspect, wherein the method exhibits an increased olefin selectivity when compared to a reference method consisting of substantially identical components except for the absence of the hydrogen stream and reacted under substantially identical conditions.

Aspect 75: The method according to any preceding aspect, wherein at least a portion of the hydrogen formed or released during a reaction step is recovered.

Aspect 76: The method according to any preceding aspect, wherein at least a portion of the recovered hydrogen is reintroduced back into the system.

Aspect 77: The method according to any preceding aspect, wherein at least a portion of the recovered hydrogen is recovered for an external facility.

Aspect 78: The method according to any preceding aspect, wherein at least a portion of the recovered hydrogen is reintroduced into the hydrogen stream for dehydrogenation.

Aspect 79: The method according to any preceding aspect, wherein at least a portion of the recovered hydrogen is used as a stripping gas.

Aspect 80: The method according to any preceding aspect, wherein at least a portion of the recovered hydrogen is used in a reverse water gas shift reaction to produce syngas.

Aspect 81: The method according to any preceding aspect, wherein the catalytic reactor is a fluidized or moving bed reactor, and wherein at least a portion of the hydrogen formed or released during a reaction step is recovered and used as a catalyst lift gas.

Aspect 82: The method according to any preceding aspect, wherein the catalyst lift gas further comprises natural gas.

Aspect 83: The method according to any preceding aspect, wherein the catalyst exhibits higher active sites stability during the course of reaction and continuously reduces the active metal sites and maintains catalytic activity without contributing to secondary reactions.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. Unless indicated otherwise, percentages referring to composition are in terms of wt %.

There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

In the following examples, performance of catalysts were investigated using an exemplary method for direct light alkane dehydrogenation co-injected with hydrogen in accordance with the method disclosed herein. The hydrocarbon feed comprised pure propane or iso-butane. For each trial, measured amounts of catalyst samples were loaded into the reactor such to maintain the desired WHSV with varying hydrogen/hydrocarbon molar ratios. The catalysts were reduced under flowing H₂ (1-2 milliliters per gram per minute (ml/g/min)) at 500-650° C. for between 0.1-1 hr. Reaction feed streams composed of H₂ and hydrocarbon in different molar ratios (0.01-0.8) were evaluated. Delivery of the determined amount of hydrocarbon feed and/or hydrogen was controlled by mass flow controllers.

Multiple experimental runs were conducted using an isothermal fixed bed micro-reactor (0.5 inch (1.27 centimeter (cm) internal diameter) and a bench scale fluidized bed reactor (FBR) (2.5 inch (6.35 cm) internal diameter) to ensure satisfactory intrinsic kinetics and differential data analysis.

The composition of the product stream was analyzed using an online gas chromatography system equipped with a FID detector. The reactor outlet was attached to the GC system, such that the product stream exiting the reactor outlet was injected into the GC system for direct determination of the product distribution profile. All the values were calculated in weight.

All calculations were performed based on a pure hydrocarbon feedstock composition. The standard conversion value was determined according to the formula:

(% Conversion)=(Alkane in Feed-Alkane in Product/Alkane in Feed)×100.

The standard selectivity value was determined according to the formula:

(% Selectivity)=(% of desired product/Conversion)×100.

### Example 1

For Example 1, catalyst A (Cr₂O₃/Al₂O₃) was prepared following standard catalyst preparation procedures using Al₂O₃ as the support, and impregnation with chromium. The final catalyst had a content of 17 wt. % Cr, which was confirmed with XRF. Catalyst performance was then tested using a pure iso-butane feed with and without co-injection of hydrogen as described herein. For this exemplary example, the selected hydrogen co-injection sites on the FBR reactor are depicted in FIG. 1 with the arrows adjacent to 1 and 9, which corresponds to the feed and stripping gas lines. All reactions in Example 1 were run in a fluidized bed reactor, maintained at LHSV 400/hr⁻¹ and 590° C. for 10 minutes (min). Without wishing to be bound by a particular theory, this method will operate similarly in fixed bed reactors.

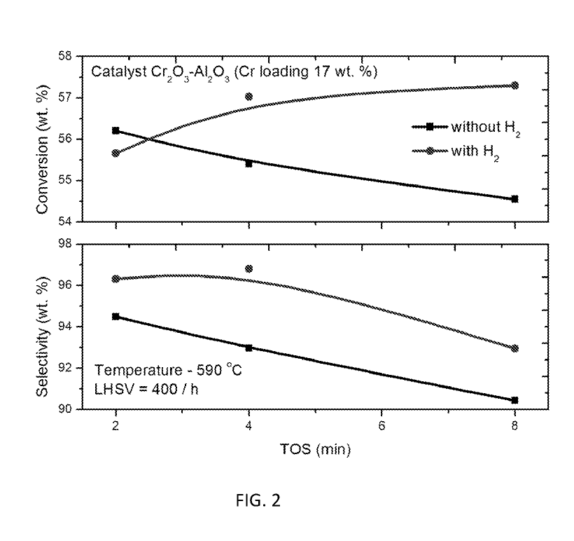

Results of catalyst performance with and without hydrogen are shown in FIG. 2. As the data demonstrates, the inventive methods exhibited higher selectivity and conversion of butane on catalyst A. The results suggest that dehydrogenation over Cr-based catalyst is enhanced by the co-injection of hydrogen.

### Example 2

For Example 2, catalyst B (Pt-Sn/Al₂O₃) was prepared following standard catalyst preparation protocols procedures using Al₂O₃ as the support. Pt and promoter Sn were sequentially impregnated to yield a final catalyst content 0.5 wt % and 1 wt %, respectively based on their respective salts. After each incorporation, the catalyst was dried at a temperature of 100° C. for 4 hours and calcined at a temperature of 500° C. for 4 hours. Prior to the reaction, catalyst B was de-chlorinated with steam at 500° C. for 4 hours and then activated in hydrogen environment at 500° C. for 10 h (8 ml/min). Measured amount of catalyst B was employed in a micro-reactor to achieve the desired WHSV (6 h⁻¹). All feedstock used in Example 2 comprised propane feedstock (99.5% pure), and when when present, hydrogen co-injected to achieve hydrogen/propane molar ratio of 0.2. The reaction temperature in the reactor was 590° C. Data for propane dehydrogenation using catalyst B with and without hydrogen co-injection is provided in Table 1. As the data shows, the inventive methods utilizing co-injection of hydrogen also exhibited higher selectivity and conversion of propane to propylene over catalyst B, suggesting the herein described dehydrogenation method comprising co-injection of hydrogen exhibits the same performance enhancements using a Pt-based catalyst.

TABLE 1*

| | 590° C., 1 atm, WHSV = 6 h⁻¹ | | | | | |
|---|---|---|---|---|---|---|
| | TOS* = 0 h | | TOS = 1 h | | TOS = 2 h | |
| | | H₂/C₃H₈ | | H₂/C₃H₈ | | H₂/C₃H₈ |
| | No H₂ | molar | No H₂ | molar | No H₂ | molar |
| Catalyst B | Injection | ratio = 0.2 | Injection | ratio = 0.2 | Injection | ratio = 0.2 |
| Propylene Selectivity (%) | 66.77 | 81.24 | 87.15 | 92.31 | 88.32 | 94.01 |
| Total Olefin Selectivity (%) | 75.31 | 86.21 | 91.56 | 94.12 | 91.88 | 94.56 |
| Propane Conversion (%) | 34.63 | 41.07 | 24.57 | 29.87 | 21.96 | 24.13 |
| Propylene Yield (%) | 23.13 | 33.36 | 21.42 | 27.57 | 19.4 | 22.68 |

*TOS = Time on stream, and TOS = 0 h was conducted at 1 min.

1. A method for dehydrogenation of an alkane comprising:
(a) co-injecting a hydrogen feed stream with a hydrocarbon feed stream comprising at least one C₂ to C₅ alkane into a non-membrane dehydrogenation catalytic reactor comprising a chromium-based catalyst or platinum-based catalyst; and
(b) producing at least one dehydrogenated alkane product;
wherein the reactor is run under substantially non-oxidative conditions.

2. The method of claim 1, wherein the at least one 1 dehydrogenated alkane product comprises a corresponding alkene product.

3. (canceled)

4. (canceled)

5. (canceled)

6. The method according to claim 1, wherein the dehydrogenated alkane product comprises ethene, propene, n-butene, iso-butene, n-pentene, or iso-pentene, or a combination thereof.

7. (canceled)

8. The method according to claim 1, wherein the dehydrogenated alkane product is substantially free of water or carbon dioxide.

9. The method according to claim 1, wherein the reactor is run under fully non-oxidative conditions.

10. (canceled)

11. (canceled)

12. (canceled)

13. The method according to claim 1, wherein the hydrogen feed stream is contacted with the hydrocarbon feed stream prior to preheating of the hydrocarbon feed stream, during preheating of the hydrocarbon feed stream, or after preheating of the hydrocarbon feed stream.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. The method according to claim 1, wherein the method comprises multiple catalytic reactors.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. The method according to claim 1, wherein the catalyst bed comprises heat generating material.

56. The method according to claim 1, wherein the heat generating material is interspersed among, physically mixed with, or inserted between inert materials.

57. The method according to claim 1, wherein the heat generating material forms a layer on top, bottom, or in the center of the catalyst bed.

58. The method according to claim 1, wherein the heat generating material comprises at least 10 wt % of the catalyst bed.

59. (canceled)

60. (canceled)

61. (canceled)

62. (canceled)

63. (canceled)

64. (canceled)

65. (canceled)

66. (canceled)

67. The method according to claim 1, wherein the hydrogen feed stream and hydrocarbon feed stream is in a liquid phase, or a vapor phase, or a combination thereof.

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. The method according to claim 1, wherein the hydrogen feed stream is contacted with a hydrocarbon feed stream between catalytic dehydrogenation reaction zones.

85. The method according to claim 1, wherein the catalyst is physically interspersed with the inert materials, or located between inert materials.

86. The method according to claim 1, wherein the heat generating material comprises at least 25 wt % of the catalyst bed.

87. The method according to claim 1, wherein the heat generating material comprises at least 50 wt % of the catalyst bed.

88. The method according to claim 1, wherein the heat generating material comprises at least 60 wt % of the catalyst bed.

89. The method according to claim 1, wherein at least a portion of the recovered hydrogen is used as a stripping gas.

90. The method according to claim 1, wherein the catalytic reactor is a fluidized or moving bed reactor, and wherein at least a portion of the hydrogen formed or released during a reaction step is recovered and used as a catalyst lift gas.

91. The method according to claim 1, wherein the catalyst lift gas further comprises natural gas.
